(19) United States
(12) Patent Application Publication
(10) Pub. No.: US 2006/0210650 A1
(43) Pub. Date: Sep. 21, 2006

(54) Supplemental dietary composition for promoting weight loss

(76) Inventors: Paul T. Gardiner, Mississauga (CA); Marvin A. Heuer, Mississauga (CA)

Correspondence Address: KENYON & KENYON LLP, ONE BROADWAY, NEW YORK, NY, 10004, US

(21) Appl. No.: 11/304,195

(22) Filed: Dec. 14, 2005

Related U.S. Application Data
(60) Provisional application No. 60/635,867, filed on Dec. 14, 2004.

(51) Int. Cl. A61K 38/17; A61K 33/06; A61K 31/59; A61K 31/555; A61K 31/045; A61K 36/254; A61K 36/82; A61K 36/87

(52) U.S. Cl. 424/682; 424/728; 424/729; 424/766; 424/769; 514/184; 514/2; 514/167; 514/574; 514/724

(57) ABSTRACT

Compositions, and methods for administering the same to humans, are provided for the promotion of healthy fast weight loss while providing an excellent source of calcium and containing antioxidants. The compositions preferably comprise three sources of calcium and include at least one of Calcium Carbonate, Vitamin D, caffeine-free Green Tea, Garcinia Cambogia Extract, Gymnema Sylvestre Extract, Ginseng, Grape Skin Extract, Chromium Polynicotinate, and Policosanol. The supplemental dietary composition may be suitable for individuals on a calorie-restricted diet and exercise program.

### RELATED APPLICATIONS

The application is related to and claims benefit of priority to U.S. Provisional Patent Application Ser. No. 60/635,867, entitled “Supplemental Dietary Compositions for Promoting Weight Loss,” filed Dec. 14, 2004, the disclosure of which is hereby fully incorporated by reference.

### FIELD OF THE INVENTION

The present invention relates to a supplemental dietary composition that at least one of promotes healthy weight loss, is an excellent source of calcium, and contains antioxidants, and to a method of promoting same by consuming the supplemental composition.

### SUMMARY OF THE INVENTION

The present invention provides for supplemental dietary composition including one or more sources of calcium, preferably from more than one source and most preferably from three sources, garcinia cambogia and/or gymnema sylvestre extracts, wherein the supplemental dietary composition promotes healthy weight loss, provides an excellent source of calcium, and provides antioxidants. In a preferred embodiment, the composition includes calcium carbonate.

The present invention also provides, by the consumption of the supplemental composition, a method of promoting healthy weight loss, providing an excellent source of calcium, and providing antioxidants.

### DETAILED DESCRIPTION OF THE INVENTION

As used herein, “an effective amount” refers to an amount effective for inducing weight loss when administered, e.g., as a serving three times a day over a period of time, e.g., several week(s), and most preferably for at least eight weeks. An effective amount of the supplemental dietary composition is preferably from about 0.1 g to about 10 g of the supplemental dietary composition. More preferably, an effective amount of the composition comprises from about 1 g to about 5 g of the supplemental dietary composition. Most preferably, an effective amount of the supplemental dietary composition comprises about 1.2 g to 4.5 g of the nutritional composition.

The present invention, according to various embodiments thereof, is directed to a supplemental dietary composition that includes, e.g., one or more of calcium, garcinia cambogia and gymnema sylvestre extracts. Preferably, the calcium is provided from more than one source and most preferably is provided from three sources. The supplemental composition according to this invention promotes healthy weight loss, provides an excellent source of calcium, and provides antioxidants. In preferred embodiments, the compositions include calcium carbonate.

According to various embodiments of the present invention, the supplemental composition may be consumed in any form. For instance, the dosage form of the supplemental composition may be provided as, e.g., a capsule, a tablet, a caplet, a liquid beverage, a powder beverage mix, a dietary gel, or as a ready-to-eat bar or drink product. The most preferred dosage form is caplet.

Furthermore, the dosage form of the supplemental composition in accordance with this embodiment may be provided in accordance with customary processing techniques for herbal and/or dietary supplements in any of the forms mentioned above. For instance, in the embodiment in which the supplemental composition is provided is a capsule, the active ingredients may be suitably processed and encapsulated into capsules, e.g., cellulose, gelatin, etc., or compressed into caplets with cellulose, croscarmellose sodium, stearic acid, magnesium stearate, silica and other with suitable excipients. Those of skill in the art will appreciate that the supplemental composition may contain a variety of excipients.

In one embodiment of the present invention, which is set forth in greater detail in, e.g., Example 1 below, there is provided a supplemental dietary composition for promoting healthy weight loss that includes at least calcium carbonate. According to this embodiment, the supplemental dietary composition provides a method of promoting and supporting healthy weight loss, of providing an excellent source of calcium, and of providing antioxidants. In this manner, the consumption of the supplemental composition provides for a method for promoting and supporting healthy weight loss, for providing an excellent source of calcium, and also for providing antioxidants. According to the example embodiment set forth below, the supplemental composition may be provided and consumed in any form, e.g., a capsule, a tablet, a caplet, a liquid beverage, a powder beverage mix, a dietary gel, or as a ready-to-eat bar or drink product. However, the preferred form for consumption of the supplemental composition in accordance with the example embodiments set forth below is a caplet.

Furthermore, the dosage form of the supplemental composition in accordance with the example embodiments set forth below may be provided in accordance with customary processing techniques for herbal and/or dietary supplements, wherein the active ingredients are suitably processed and compressed into, e.g., tablets and/or caplets, with suitable excipients. Those of skill in the art will appreciate that the supplemental compositions set forth in the example embodiments below may contain a variety of, and any number of different excipients.

Vitamin D

Vitamin D is a steroid hormone, known to have an important role in regulating body levels of calcium and phosphorus, and in mineralization of bone. Vitamin D is particularly important for the absorption of calcium from the stomach and for the functioning of calcium in the body. Thus, vitamin D is particularly important for strong bones and teeth.

Vitamin D is also known as cholecalciferol, which is generated in the skin of animals when light energy is absorbed by a precursor molecule 7-dehydrocholesterol. Cholecalciferol is used as a dietary supplement to treat and/or prevent low levels of vitamin D in the body.

Although the present invention is not to be limited by any theoretical explanation, it is believed that vitamin D is highly involved in mineral metabolism, as well as bone growth and function. It has been shown that vitamin D effectively facilitates intestinal absorption of calcium, and stimulates absorption of phosphate and magnesium ions. In the absence of vitamin D, dietary calcium may not be absorbed efficiently. Vitamin D stimulates the expression of a number of proteins involved in transporting calcium from the lumen of the intestine, across the epithelial cells and into blood.

As used herein, a serving of the supplement comprises from about 0.000001 g to about 1 g of vitamin D per serving. More preferably, a serving of the supplement comprises about 0.0000033 g or 132 IU of vitamin D per serving.

Calci-Triplex™

Calci-Triplex™ is proprietary dietary ingredient blend which comprises Calcium, Calcium Carbonate, Calcium Hydroxycitrate (from, e.g., Super CitriMax®) and Whey Mineral Complex (currently from TruCal® D50). Moreover, Calci-Triplex™ comprises dairy-source ingredients high in calcium and other important bone-building mineral such as phosphorus, magnesium, potassium, zinc, copper, and iron. As used herein, serving of the supplement comprises from about 0.1 g to about 5 g of Calci-Triplex™. More preferably, a serving of the supplement comprises from about 0.1 g to about 2 g of Calci-Triplex™ per serving.

Although the following example illustrates the practice of the present invention in one embodiment, the example should not be construed as limiting the scope of the invention. Other embodiments will be apparent to one skilled in the art from consideration of the specification and the following examples.

Calcium Carbonate

Calcium carbonate is a colorless or white crystalline compound, CaCO₃, occurring naturally as chalk, limestone, marble, and other forms and used in a wide variety of manufactured products including dentifrices and medicines.

Although the present invention is not to be limited by any theoretical explanation, it is believed that increased calcium intake has a significant effect on body weight and body fat. Studies show a consistent effect of higher calcium intakes, expressed as lower body fat and/or body weight, and reduced weight gain at midlife. Specifically, calcium may assist in weight loss, weight management, regulation of metabolism, and the breakdown of fat. Studies show that increasing dietary calcium speeds up weight and fat loss. In a comparison trial, both the diets, either high in calcium or low in calcium, produced significant weight and fat loss. Weight and fat loss with respect to the high dairy diet were approximately 2-fold higher (p<0.01), and loss of lean body mass was markedly reduced (p<0.001) compared with the low dairy diet. This study purports to show that employing calcium-rich foods in isocaloric diets reduced adiposity and improved metabolic profiles in obese African Americans (Zemel et al., 2005). Additionally, calcium carbonate facilitates the prevention and treatment of calcium deficiencies, and thereby facilitates bone formations and maintenance.

Furthermore, Zemel et al., 2005b, purports to show that an isocaloric substitution of yogurt (diary) for other foods can significantly reduce central adiposity during energy restriction and therefore augment fat loss. This was purportedly shown by the use of control subjects (400-500 mg Ca/day), or yogurt diet (1100 mg Ca/day) with the dairy macronutritents controlled to the United States average values. After 12 weeks, fat loss was markedly increased in the yogurt group, and mean tissue loss was also increased in the yogurt group by 31%. Compared to the control group, an 81% increase in trunk fat loss was also purported to be seen in the yogurt group, this resulted in decrease in waist diameter in yogurt group (Zemel et al., 2005b). Moreover, in 2004, Zemel and his colleagues purported to show that calcium and dairy supplementation accelerated fat and weight loss during a 24 week controlled study. Subjects on high-dairy diet (1200-1300 mg Ca/day) reportedly lost 70% more weight than those on the standard diet of 400-500 mg Ca/day and subjects on the calcium enriched, but non-dairy diet lost on 26% more weight than those on the standard diet. Therefore, this study purports to show that calcium supplementation significantly increases weight loss and that calcium supplementation from diary sources exerts a substantially greater effect of the weight and fat loss (Zemel et al., 2004).

U.S. Pat. No. 6,384,087, incorporated herein by reference in its entirety, purports to disclose a method of inducing the loss of adipose tissues by providing a high calcium diet in the form of dairy products or as calcium carbonate. The patent purports that the invention acts via stimulating lipolysis, inhibiting lipogenesis, and increasing the expression of uncoupling protein 2 in white adipose tissue. The patent also purports to disclose a method for increasing the core temperature of an individual.

As used herein, a serving of the supplement comprises from about 0.01 g to about 10 g of calcium carbonate per serving. More preferably, a serving of the supplement comprises from about 0.05 g to about 2 g of calcium carbonate per serving. Most preferably, a serving of the supplement comprises about 0.558 g of calcium carbonate per serving.

Phosphorus

Phosphorus is known to help conserve calcium by decreasing the amount of calcium that is lost in the urine. Moreover, in bone, phosphorus is part of calcium phosphate which is a major contributor to the formation of bone, thereby, leading to the development of strong and healthy bones and teeth (Groff, 1999).

Although not wishing to be bound by theoretical explanation, it is believed that phosphorus is desirable for the control of excretion of calcium, thereby leading to weight loss, as well the maintenance and development of strong bone and teeth. In the absence of phosphorus, calcium is excreted in the urine more readily.

As used herein, a serving of the supplement comprises from about 0.001 g to about 1 g of phosphorus per serving. More preferably, a serving of the supplement comprises from about 0.01 g to about 0.05 g of phosphorus per serving currently from TruCal® D50 as noted below.

Chromium Polynicotinate

Chromium plays a role in the metabolism of glucose, and is necessary for energy production. Since this mineral assists in the production of insulin, it helps to stabilize blood sugar levels and can be beneficial both for people with hypoglycemia and diabetes. It is also important to the synthesis of cholesterol, fats, and proteins.

Chromium polynicotinate may be more effective than other types of chromium, as it binds the elemental chromium to niacin (vitamin B-3), thus also known as niacin-bound chromium. This provides a biologically active form of chromium, which is more absorbable in the body.

Although the present invention is not to be limited by any theoretical explanation, it is believed that chromium is mainly used to stabilize blood sugar. The major role for chromium with respect to weight loss is to correct insulin resistance caused by a deficiency in chromium. Studies have shown that chromium is at the heart of a biologically active complex called Glucose Tolerance Factor or “GTF”, which is responsible for potentiating (increasing) the action of insulin in the body. Insulin is a vital hormone responsible for transporting glucose inside cells and maintaining proper blood sugar and blood lipid (cholesterol) levels in the body. Chromium helps insulin metabolize fat, turn protein into muscle, and convert sugar into energy.

As used herein, a serving of the supplement comprises from about 0.00001 g to about 1 g of chromium polynicotinate per serving. More preferably, a serving of the supplement comprises from about 0.0001 g to about 0.5 g of chromium polynicotinate per serving. Most preferably, a serving of the supplement comprises about 0.00133 g chromium polynicotinate (0.000133 of elemental chromium) per serving.

Potassium

Potassium is known to have an effect on the urinary excretion of calcium, in that it decreases the excretion of calcium. Deficiencies in potassium can be manifested in symptoms such as muscle weakness, mental apathy, and bone fragility (Groff, 1999).

Although not wishing to be bound by theoretical explanation, it is believed that potassium is desirable for the retention of calcium, leading to weight loss, and for the promotion of weight loss as well as strong, healthy bone and teeth. As used herein, a serving of the supplement comprises from about 0.1 g to about 2 g of potassium per serving. More preferably, the supplement comprises from about 0.2 g to about 0.7 g of potassium per serving from, e.g., Super CitriMax®.

A. TrimSource™

TrimSource™ is a proprietary dietary ingredient blend which is comprised of Garcinia Cambogia Extract from, e.g., Super CitriMax® (standardized for 60% (−) hydroxycitric acid), Gymnema Sylvestre Extract (standardized for 25% gymnemic acids) and Grape Skin Extract (standardized for 20% proanthocyanidins). As used herein, a serving of the supplement comprises from about 0.01 g to about 10 g of TrimSource™ per serving. More preferably, a serving of the supplement comprises from about 0.05 g to about 6 g of TrimSource™ per serving.

A.1. Garcinia Cambogia (Hydroxycitric Acid and/or Various Salts)

According to various embodiments of the present invention, the supplemental dietary composition includes garcinia cambogia alone or as various salts e.g., mono-, di-, tri-, or poly salts of garcinia cambogia (standardized, but not limited to e.g., 50%; 60% hydroxycitric acid). Advantageously, these ingredients may be provided as, but not limited to, e.g., full strength Super CitriMax® from InterHealth N.I. (garcinia cambogia and its calcium/potassium salts).

Super CitriMax® is an all-natural, safe and effective plant extract used as diet ingredients. Extracted from the South Asian fruit Garcinia cambogia, Super CitriMax® contains standardized levels of (−) hydroxycitric acid (HCA), which has been clinically shown to suppress appetite and inhibit fat production, without stimulating the central nervous system. More recently, researchers have shown that clinical strength doses of Super CitriMax®, as aforementioned in TrimSource™, may be an even more powerful, all-natural diet aid for reducing and/or maintaining weight.

Garcinia cambogia, also known as Garcinia cambogia or Mangosteen oil tree, contains a biologically active compound, which is known to inhibit the synthesis of lipids and fatty acids and lower the formation of Low Density Lipoproteins LDL and triglycerides. Garcinia cambogia also contains significant amounts of Vitamin C.

Although the present invention is not to be limited by any theoretical explanation, it is believed that garcinia cambogia may inhibit the conversion of excess calories to body fat, and may aid in the control of obesity and cholesterol.

Additionally, appetite may also be suppressed by the promotional effect of garcinia cambogia on the synthesis of glycogen. Glycogen is the stored form of glucose, one of the body's primary sources of energy. Increased glycogen production and storage is the body's normal way of signaling satiety to the brain.

Studies have shown that hydroxycitric acid, the principal active ingredient from garcinia cambogia fruit, effectively curbs or controls appetite and inhibits fatty acid synthesis (production) in animals.

Moreover, in pilot study (−) hydroxycitric acid alone or in combination with niacin-bound chromium and Gymnema Sylvestre it was found that HCA reduces appetite, inhibits fat synthesis and decreases body weight without stimulating the central nervous system (Preuss et al., 2004). Moreover, in a second study by Preuss et al., 2004b, it was confirmed that HCA alone, or in combination with Gymnema sylvestre and niacin-bound chromium has an effect of inducing weight loss in moderately obese subjects. Groups treated with either HCA, or HCA with Gymnema sylvestre and niacin-bound chromium showed a 5-6% decrease in BMI and body weight over the course of the 8-week study compared to a control group (Preuss at al., 2004b). Additionally, these subjects also showed a decrease in LDL-cholesterol, triglycerides and serum leptin levels, while High-Density Lipoproteins and excretion of urinary fat metabolites increased in both treatment groups (Preuss at al., 2004b). These two studies both show that HCA, alone or in combination with Gymnema sylvestre and niacin-bound chromium can serve as effective and safe weight loss aids while promoting healthy blood lipid levels.

U.S. Pat. No. 6,638,542, incorporated by reference in its entirety, discloses a method of manufacture and of Procyanidin, optionally combined with hydroxycitric acid, is used in a method for the reduction of appetite in a mammal.

U.S. Pat. No. 6,383,482, incorporated by reference in its entirety, discloses a formulation containing green tea extract, hydroxycitric acid, 5-hydroxytryptophan, glucomannan, chromium picolinate, and Lactobacillus acidophilus for the purposes of weight loss via increasing metabolic rates, suppressing appetite and helping to burn fat.

U.S. Pat. No. 5,783,603, incorporated by reference in its entirety, discloses a method of suppressing appetite and causing weight loss by administering hydroxycitric acid in the form of its potassium salt extracted from Garcinia fruit. Moreover, methods of inhibiting cytoplasmic citric lyase and increasing fat metabolism in a patient are also described.

As used herein, a serving of the supplement comprises from about 0.01 g to about 10 g of garcinia cambogia per serving. More preferably, a serving of the supplement comprises from about 0.05 g to about 2 g of garcinia cambogia per serving. Most preferably, a serving of the supplement comprises about 1.55550 g of garcinia cambogia per serving. Approximately 60% of the garcinia cambogia may be hydroxycitric acid.

A.2. Gymnema Sylvestre

Gymnema sylvestre, is comprised in TrimSource™, also known as Gurmar and Meshashringi, has long been known to have hypoglycemic (blood sugar-lowering) properties. The leaf extracts contain gymnemic acid, which inhibits hyperglycemia and also acts as a cardiovascular stimulant.

Although the present invention is not to be limited by any theoretical explanation, it is believed that gymnema raises insulin levels by regeneration of the cells in the pancreas that secrete insulin. Studies have shown that gymnema also improves uptake of glucose into cells by increasing the activity of the glucose utilizing enzymes, and prevents adrenaline from stimulating the liver to produce glucose, thereby reducing blood sugar levels. Gymnema is also noted for lowering serum cholesterol and triglyceride levels, and for its ability to suppress appetite and cravings for sweetness due to its ability to abolish the taste of sugar.

As used herein, a serving of the supplement comprises from about 0.01 g to about 10 g of gymnema sylvestre per serving. More preferably, a serving of the supplement comprises from about 0.05 g to about 2 g of gymnema sylvestre per serving. Most preferably, a serving of the supplement comprises about 0.133 g of gymnema sylvestre per serving comprising 25% gymnemic acids.

A.3. Grape Skin Extract

Grape skin extract, included in TrimSource™, is derived from the skin of grapes and is a concentration of the pure oligomeric proanthocyanidins found in grape skins. Grape skin is rich in proanthocyanidin flavonoids, which have demonstrated a number of important biochemical properties such as acting as free-radical scavengers, thereby helping in resistance to blood vessel damage and skin damage, inhibiting inflammatory enzymes, and inhibiting histamine formation. Grape skin extract is mainly used as a supplement for anti-oxidant health products.

Although the present invention is not to be limited by any theoretical explanation, it is believed that grape skin extract may improve circulation and capillary activity because grape skin extract contains natural free radical scavengers and bioflavonoid complexes which are rapidly absorbed and distributed throughout the body.

Studies have shown that grape skin extract may work with glutathione to restore oxidized vitamin C, cross the blood barrier to provide potent antioxidant protection to the brain and spinal nerves, remove lipfuscin and prevent its formation in the brain and heart, thereby conferring anti-aging benefits, bind to collagen fibers, thereby increasing elasticity and flexibility in muscles, tendons, and ligaments, and act as a smooth muscle relaxant in blood vessels, to combat hypertension. Additionally, studies have shown that, in vitro, the antioxidant power of grape skin extract may be 50 times greater than vitamin E and 20 times greater than vitamin C.

As used herein, a serving of the supplemental dietary composition preferably comprises from about 0.01 g to about 5 g of grape skin extract per serving. More preferably, a serving of the supplemental composition comprises from about 0.05 g to about 2 g of grape skin extract per serving. Most preferably, a serving of the supplemental composition comprises 0.02 g of grape skin extract per serving.

B. SourceCal™

SourceCal™ is a proprietary dietary ingredient blend which comprises Caffeine-free Green Tea Extract (standardized for 95% polyphenols [70% catechins (45% epigallocatechin gallate, 10% epicatechin gallate, 8% epigallocatechin, 4% epicatechin, 3% gallocatechin gallate—90 mg EGCG), Whey mineral complex (currently from TruCal® D50 also supplying phosphorus and a source of Calcium), American Ginseng (standardized for 3% ginsenosides [as Panax quinquefolius]), and Policosanol. As used herein, a serving of the supplement comprises from about 0.001 g to about 5 g of SourceCal™. More preferably, a serving of the supplement comprises from about 0.1 g to about 1 g of SourceCal™ per serving.

B.1. Caffeine Free Green Tea

Green tea (camellia sinesis), which is included in SourceCal™, is a naturally rich source of antioxidants and contains natural antioxidant compounds known as polyphenols. As used herein, green tea preferably refers to 45% extract, caffeine-free.

Although the present invention is not to be limited by any theoretical explanation, it is believed that green tea extract increases metabolic rate and fat oxidation, and decreases appetite. Studies have shown that green tea extract increases the metabolic rate by up to 4%, and the increase in fat oxidation was higher than in subjects who used a placebo or caffeine. In addition, this stimulation of thermogenesis, resulting from an increase in metabolic rate and fat oxidation by the green tea extract was not accompanied by an increase in heart rate. Other studies have found that green tea caused rats to lose up to 21% of their body weight due to a reduction in appetite. The rats consumed up to 60% less food after seven days of daily injections with a green tea extract.

As used herein, a serving of the supplement comprises from about 0.01 g to about 10 g of green tea extract per serving. More preferably, a serving of the supplement comprises from about 0.05 g to about 2 g of green tea extract per serving. Most preferably, a serving of the supplement comprises about 0.2 g of green tea extract per serving.

B.2. Ginseng

As used herein, Ginseng preferably refers to American Ginseng (Panax quinquefolium L.), or Korean Ginseng (Panax Ginseng) or Siberian Ginseng (Eleutherococcus senticosus) and derivatives thereof is comprised in SourceCal™. Most preferably ginseng refers to American Ginseng (Panax quinquefolium L.) and derivatives thereof.

Although the present invention is not to be limited by any theoretical explanation, it is believed that that ginseng stimulates the production of nitric oxide.

As used herein, a serving of the supplemental dietary composition preferably comprises from about 0.001 g to about 0.5 g of American ginseng or Korean ginseng per serving. More preferably, a serving of the supplemental composition comprises from about 0.01 g to about 0.1 g of American ginseng per serving. Most preferably, a serving of the supplemental composition comprises 0.01000 g of American ginseng per serving.

B.3. Policosanol

Policosanol refers to a unique natural product, a mixture of solid long chain alcohols (waxes) derived from sugar cane wax. The long-chain alcohols found in policosanol are present in many foods of plant origin. Supplemental policosanol is typically extracted from sugar cane wax or beeswax.

Although the present invention is not to be limited by any theoretical explanation, it is believed that policosanol is effective at reducing cholesterol levels. Research has also shown that policosanol reduces cardiac risk factors by inhibiting platelet aggregation, and inhibiting the development of atherosclerosis. In addition, other studies have shown policosanol is a very promising phytochemical alternative to classic lipid-lowering agents such as statins.

As used herein, a serving of the supplement comprises from about 0.0001 g to about 5 g of policosanol per serving. More preferably, a serving of the supplement comprises from about 0.0005 g to about 2 g of policosanol per serving. Most preferably, a serving of the supplement comprises about 0.001 g policosanol per serving.

### EXAMPLE 1

A serving of the supplemental dietary composition may include, but is not limited to, the following ingredients. Suggested directions are also provided.

| Ingredient # | Ingredient | | Amount |
|---|---|---|---|
| 1 | Vitamin D | | 132 IU |
| 2 | Calcium Carbonate‡‡ | | 404 mg (as |
| 3 | Calcium | | elemental |
| | Hydroxycitrate***‡‡ | | calcium) |
| 4 | Whey Mineral Complex‡‡ | | |
| 5 | Phosphorus | | 13.7 mg |
| | | | (elemental |
| | | | Phosphorus) |
| 6 | Chromium Polynicotinate** | | 133 mcg |
| | | | (elemental |
| | | | Chromium) |
| 7 | Potassium*** as | | 218 mg |
| | hydroxycitrate | | (elemental |
| | | | Potassium) |
| 8 | TrimSource ™ | Garcinia | 1.7 g |
| | | Cambogia | |
| | | Extract*** | |
| | | Gymnema | |
| | | Sylvestre | |
| | | Extract | |
| | | Grape Seed | |
| | | Extract | |
| 9 | SourceCal ™ | Caffeine-free | 276 mg |
| | | Green Tea | |
| | | Extract | |
| | | Whey Mineral | |
| | | Complex‡‡ | |
| | | American | |
| | | Ginseng | |
| | | Policosanol | |

**ChromaTech ™
***Super CitriMax ™ containing double salts of garcinia cambogia.
‡‡Calci-Triplex ™

A serving of the dietary supplement, take 3 caplets with an 8 oz. glass of water 3 times daily, approximately 30 to 60 minutes before meals (preferably with breakfast, lunch and dinner). Do not exceed 3 caplets in a 4-hour period and/or 9 caplets in a 24-hour period. To assess individual tolerance, the following chart may be provided:

| | |
|---|---|
| Day 1 to Day 3 | 1 caplet, 3X daily |
| Day 4 to Day 7 | 2 caplets, 3X daily |
| Day 8 and beyond | 3 caplets, 3X daily |

While the present invention has been particularly described, in conjunction with specific example embodiments thereof, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

What is claimed:

1. A supplemental dietary composition for at least one of promoting weight loss, providing a source of calcium and providing antioxidants, the composition comprising at least one of calcium and/or a salt of calcium and garcinia cambogia extract.

2. The supplemental dietary composition of claim 1, wherein the calcium is calcium carbonate and garcinia cambogia is standardized to about 60% hydroxycitric acid.

3. The supplemental dietary composition of claim 1, further comprising at least one of Garcinia Cambogia Extract, Gymnema Sylvestre Extract, and Grape Skin Extract.

4. The supplemental dietary composition of claim 1, further comprising mono-, di-, tri- or poly salts of garcinia cambogia comprising at least one of Calcium Carbonate, Calcium Hydroxycitrate, and Potassium hydroxycitrate.

5. The supplemental dietary composition of claim 1, further comprising Calcium Carbonate, Calcium Hydroxycitrate and Whey mineral complex.

6. The supplemental dietary composition of claim 1, further comprising chromium polynicotinate.

7. The supplemental dietary composition of claim 1, further comprising at least one of caffeine-free Green Tea, American Ginseng, Whey mineral complex and Policosanol.

8. The supplemental dietary composition of claim 1, further comprising Vitamin D.

9. The supplemental dietary composition of claim 1, wherein the calcium is present in an amount effective to at least one of provide an excellent source of calcium, provide a source of vitamin D, enhance the body's natural ability to burn fat, assist in preventing weight gain, provide antioxidants, and increase fast weight loss.

10. The supplemental dietary composition of claim 1, where the supplemental dietary composition includes a time release delivery system, and wherein the timed release delivery system is one of quick release, slow release and controlled release.

11. A method for one of promoting healthy weight loss, providing a source of calcium and providing antioxidants, the method comprising the steps of: ingesting a supplemental dietary composition that includes at least one of calcium and a salt of calcium, and that further includes a source of antioxidants.

12. The method of claim 11, wherein the calcium is calcium carbonate.

13. The method of claim 11, the supplemental dietary composition further comprising at least one of Garcinia Cambogia Extract, Gymnema Sylvestre Extract, and Grape Skin Extract.

14. The method of claim 11, the supplemental dietary composition further comprised of mono-, di-, tri- or poly salts of garcinia cambogia comprising at least one of Calcium Carbonate, Calcium Hydroxycitrate, and Potassium hydroxycitrate.

15. The method of claim 11, the supplemental dietary composition further comprising Calcium Carbonate, Calcium Hydroxycitrate and Whey mineral complex.

16. The method of claim 11, the supplemental dietary composition further comprising chromium polynicotinate.

17. The method of claim 11, the supplemental dietary composition further comprising at least one of caffeine-free Green Tea, American Ginseng, Whey mineral complex and Policosanol.

18. The method of claim 11, the supplemental dietary composition further comprising Vitamin D.

19. The method of claim 11, wherein the calcium is present in an amount effective to at least one of enhance the body's natural ability to burn fat, provide an excellent source of calcium, provide a source of vitamin D, assist in preventing weight gain, provide antioxidants, and increase fast weight loss.

20. The method of claim 11, wherein the supplemental dietary composition includes a time release delivery system and wherein the timed release delivery system is one of quick release, slow release and controlled release.

21. A supplemental dietary composition that comprises:
three sources of calcium and/or salts of calcium;
Garcinia Cambogia Extract;
Gymnema Sylvestre Extract;
Grape Skin Extract;
Whey Mineral Complex;
Chromium Polynicotinate;
caffeine-free Green Tea;
American Ginseng;
Vitamin D; and
Policosanol,
wherein the supplemental dietary composition at least one of provides 100% of human daily calcium requirements, enhances the body's ability to burn fat, and preserves lean body mass.

22. The method of claim 21, wherein the garcinia cambogia extract provides hydroxycitric acid.

23. The method of claim 21, wherein the garcinia cambogia can be a mon-, di-, tri-, or poly-salt and provides hydroxycitric acid.
